(19) United States
(12) Patent Application Publication
(10) Pub. No.: US 2021/0319855 A1
(43) Pub. Date: Oct. 14, 2021

(54) MONTE CARLO METHOD FOR THE AUTOMATED AND HIGHLY EFFICIENT CALCULATION OF KINETIC DATA OF CHEMICAL REACTIONS

(71) Applicant: Covestro Intellectual Property GmbH & Co. KG, Leverkusen (DE)

(72) Inventors: Jose Gamez, Köln (DE); Matthias Leven, Köln (DE)

(21) Appl. No.: 17/284,559

(22) PCT Filed: Oct. 16, 2019
(86) PCT No.: PCT/EP2019/078111

§ 371 Date: Apr. 12, 2021

(30) Foreign Application Priority Data
Oct. 18, 2018 (EP) 18201340.9

(51) Int. Cl. G16C 20/10

(52) U.S. Cl. CPC G16C 20/10

(57) ABSTRACT

The present invention relates to a computer-implemented method for calculating transition states of a chemical reaction, and to a system for data processing comprising means for carrying out the method, to a computer program comprising instructions which cause a computer to execute the method and to the use of the computer program.

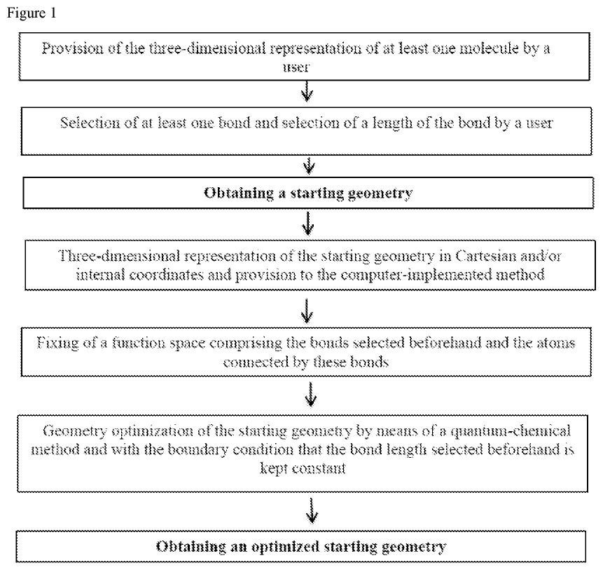

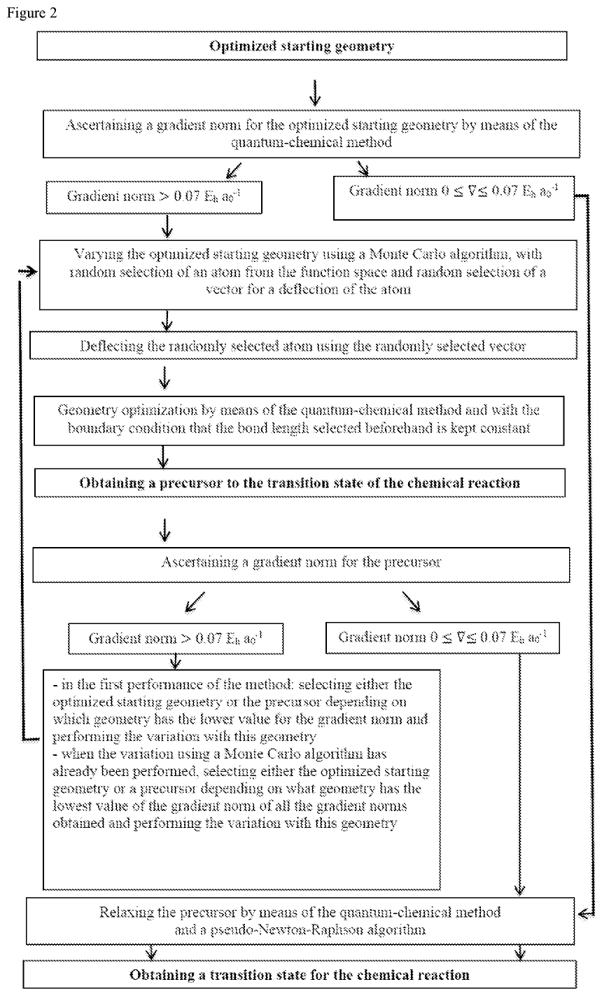

The present invention relates to a computer-implemented method of calculating transition states of a chemical reaction, and to a system for data processing comprising means of executing the method, to a computer program comprising commands that cause a computer to perform the method, and to the use of the computer program. The invention also relates to a system, a method and a means for the automated and efficient determination of kinetic data of chemical reactions.

During a chemical reaction, the geometry of the atoms involved changes and bonds are broken and new bonds are formed. There is also variation here in the energy of the atoms involved and, during the progression of the chemical reaction, it reaches a state of maximum energy, called the transition state. The transition state is a potential wall or activation barrier that divides the reactants from the products of the chemical reaction. If the activation barrier has been overcome, the product is formed. The energy during a chemical reaction can be represented with the aid of what is called a potential hypersurface which depicts the potential energy of the atoms involved in the reaction as a function of their geometry. The potential hypersurface also reflects states in which no products are formed. The direct reaction pathway in which products are formed, overcoming the transition state, can be shown as a curve in which the distances between the individual atoms of the molecules are plotted against energy. With the aid of quantum-chemical methods and mathematical approximation methods, it is possible to calculate the energy of the geometry of a molecule or a system of multiple molecules, where the function space under consideration here includes the degrees of freedom of the molecule. These methods enable the ascertaining of the geometry at the transition state and allow predictions as to the reaction kinetics of a chemical reaction.

A known method by which the geometry can be ascertained at the transition state is quasi-Newton-Raphson methods, also called pseudo-user-Raphson methods. However, the calculation of transition states with the aid of quasi-Newton-Raphson methods has some drawbacks. Firstly, quasi-Newton-Raphson algorithms do not lead to a transition state for any starting geometry of a molecule, and do so only for molecular geometries that already approximate very closely to the geometry of the transition state. Approximation is time-consuming because the approximation of a molecular geometry to the geometry of the transition state is typically effected manually, i.e. bond lengths are manually adjusted on a computer. There are also known methods in which other methods are first used to calculate potential hypersurfaces from which possible saddle points are identified. In these methods too, it is thus first necessary to ascertain a geometry already very closely approximating to the geometry of the transition state. Furthermore, quasi-Newton-Raphson methods are prone to error when they are applied to such manually selected molecular geometries.

Pseudo-Newton-Raphson-based methods often have the effect that local minima are obtained rather than the required first-order saddle points. This is because, in the theory of the Newton-Raphson-based methods, the transition state itself is expanded in a power series at the gradient=0 point. If the manually generated geometry is too far removed from the transition state itself, the prerequisite gradient≈0 is not adequately satisfied to achieve convergence. Moreover, in quasi-Newton-Raphson methods, the Hessian matrix is calculated only in the first step. In the subsequent steps, this is estimated by update algorithms, which constitutes a source of error. The further the starting structure is removed from the transition state, the more iterations or updates are required, and the greater the error or distance from the correct calculation of the Hessian matrix, and the greater the distance from convergence.

Lin et al. (“A flexible transition state searching method for atmospheric reaction systems”, Chemical Physics 450-451, 2015, p. 21-31) disclose a method of studying atmospheric chemical reactions in the gas phase. The method comprises, in a first step, a screening based on Monte Carlo methods of potential hypersurfaces by force-field methods, wherein approximate saddle point-like regions are identified. The observable used in the corresponding Monte Carlo method is the value of an energy function. Building on that, an attempt is made, by means of a quasi-Newton-Raphson method, to localize chemical transition states. With the Monte Carlo method disclosed here, it is first necessary to simulate the entire potential hypersurface before any approximate saddle point-like regions can be identified at all. Consequently, it is not possible by this Monte Carlo method to specifically optimize saddle point-like regions. For that reason, no efficient utilization of the computing resources used for localization of the saddle points required is possible. The use of the method disclosed here is limited by current computer technology to small molecules having up to 30 atoms. Moreover, mathematically highly simplified methods are used.

E. Martinez-Núñez et al. “An automated transition state search using classical trajectories initialized at multiple minima”, Phys Chem Chem Phys, 14 Jun. 2015, 17(22):14912-21; “tsscds2018: A code for automated discovery of chemical reaction mechanisms and solving the kinetics”, J. Comp. Chem., 24 Sep. 2018, 39(23)1922-1930) disclose a method of studying chemical reaction pathways. Potential hypersurfaces of chemical molecules are calculated here by mathematically simplified methods. An attempt is subsequently made to calculate the chemically relevant transition states by standard pseudo-Newton-Raphson methods. Another drawback here is that the entire potential hypersurface must first be calculated, which is time-consuming and requires computer power. Moreover, this method too can be applied only to relatively small molecules.

Jacobson et al. (“Automated Transition State Search and Its Application to Diverse Types of Organic Reactions”, J. Chem. Theory Comput. 13, 11, 5780-5797) disclose a method of automated calculation of chemical transition states. Chemical transition states are calculated here from chemical equilibrium states. This procedure requires a separate calculation of chemical equilibrium states before an interpolation of the geometries obtained, by which the transition state is approximated, can be performed. In a further step, based on this approximation, an attempt is then made to calculate a transition state geometry. The establishment of the equilibrium states required entails additional work by the person skilled in the art. The interpolation used in the method disclosed cannot be applied successfully to any desired molecular geometries.

Hu et al. (“A gradient-directed Monte Carlo method for global optimization in a discrete space: Application to protein sequence design and folding” J Chem Phys. 2009 Oct. 21; 131(15): 154117) disclose a method of calculating protein structures. In this method, foldings of protein structures are calculated by means of Monte Carlo methods. To accelerate the convergence of the Monte Carlo procedures, gradients are calculated, which affect the direction and size of deflections of the atom positions. The publication does not suggest any possible use of this method for specific calculation of saddle points.

A disadvantage of the known methods of calculating transition states is the combination of different quantum-chemical methods to calculate a potential hypersurface or a starting geometry on the one hand and to calculate the saddle point on the other hand. The potential hypersurface which is calculated by a quantum-chemical method is not necessarily suitable for calculating the saddle point by another quantum-chemical method of a different quality since different quantum-chemical methods parameters give different accuracies in the approximation of the potential hypersurface. Therefore, the saddle points obtained by these methods are frequently inaccurate or even far removed from the actual saddle point. Furthermore, the manual approximation of a molecular geometry to the geometry of the transition state is time-consuming and personnel-intensive.

There is thus a need for a method for the calculation of transition states in which a molecular geometry for a transition state of a chemical reaction can be approximated by a simple method, especially a computer-implemented method, before a calculation of the transition state can be commenced with the aid of a quantum-chemical method, especially a pseudo-Newton-Raphson method. More particularly, there is a need for a method by which transition states can be ascertained without having to calculate a potential hypersurface beforehand. Methods are used here that entail a high level of computation complexity and are not very accurate in qualitative terms. In this way, it is possible to reduce the burden on resources such as processors and storage media.

It was an object of the present invention to provide a computer-implemented method in which a geometry of a transition state is calculated by a readily employable, especially computer-implemented, method, before, in a subsequent step, the transition state is calculated with the aid of quantum-chemical methods, especially of pseudo-Newton-Raphson algorithms. More particularly, it is to be possible to ascertain a molecular geometry for a transition state of a chemical reaction without calculating the potential hypersurface of the chemical reaction beforehand.

This object was achieved by a computer-implemented method of calculating transition states of a chemical reaction, comprising the steps of

A Generating a Starting Geometry
A1 providing the three-dimensional representation of at least one molecule at ground state energy,
A2 selecting at least one bond of the at least one molecule and selecting a length of the bond, where the selected length does not correspond to the length of the bond at ground state energy of the molecule, such that a starting geometry for the chemical reaction is obtained,
A3 representing the starting geometry in three dimensions in Cartesian and/or internal coordinates,

B Ascertaining an Optimized Starting Geometry
B1 defining a function space encompassing the at least one bond from step A2 and the atoms joined by this bond,
B2 optimizing the geometry of the starting geometry on the basis of the function space selected in step B1 by means of a quantum-chemical method and with the boundary condition that the length of the at least one bond selected in step A2 is kept constant, such that an optimized starting geometry is obtained,
B3 ascertaining the gradient norm B3 for the optimized starting geometry, where the gradient norm is obtained via the first derivative of a function E=f(x) by means of the quantum-chemical method, with E=total energy of the optimized starting geometry and x=nuclear coordinates of the molecule in the optimized starting geometry,
B4.1 when the gradient norm B3 ∇ is 0≤∇≤0.07 Eₕ a₀⁻¹, classifying the optimized starting geometry as a precursor to the transition state of the chemical reaction and continuing the method with step D1, or
B4.2 when the gradient norm B3 ∇ is >0.07 Eₕ a₀⁻¹, ascertaining the precursor to the transition state of the chemical reaction proceeding from the optimized starting geometry by a method comprising the following steps:

C Ascertaining the Precursor to the Transition State of the Chemical Reaction
C1 varying the optimized starting geometry using a Monte Carlo algorithm, wherein
C1.1 at least one atom from the function space selected in step B1 is selected at random,
C1.2 a vector for a deflection of the atom chosen in step C1.1 is selected at random,
C1.3 the atom selected in step C1.1 is deflected from its position in the optimized starting geometry using the vector from step C1.2, so as to obtain a precursor to the transition state of the chemical reaction,
C2 optimizing the geometry of the precursor to the transition state by means of the quantum-chemical method and with the boundary condition that the at least one bond from step A2 has the length that was ascertained in step C1.3,
C3 ascertaining the gradient norm C3 for the precursor to the transition state from step C2, where the gradient norm is obtained via the first derivative of the function E=f(x) by means of the quantum-chemical method, with E=total energy of the precursor to the transition state and x=nuclear coordinates of the molecule in the precursor to the transition state,
C4.1 when the gradient norm C3 ∇ is 0≤∇≤0.07 Eₕ a₀⁻¹, continuing the method with step D1, or
C4.2 when the gradient norm C3 ∇ is >0.07 Eₕ a₀⁻¹, repeating steps C1 to C3 until a gradient norm C3 of 0≤∇≤0.07 Eₕ a₀⁻¹ is obtained, wherein
(a) if steps C1 to C3 have been performed once, the geometry of the precursor to the transition state is varied in step C1 when the value of its gradient norm C3 is lower than the value of the gradient norm B3 of the optimized starting geometry, or
(b) if steps C1 to C3 have been performed more than once, the geometry of the optimized starting geometry or that precursor to the transition state from the preceding repetitions that has the lowest value for the gradient norm C3 or B3 compared to all the gradient norms C3 and B3 previously obtained is varied in step C1,

D Ascertaining the Transition State
D1 relaxing the precursor from step C4.1 or the precursor from step B4.1 by means of the quantum-chemical method and a pseudo-Newton-Raphson algorithm, such that the transition state is obtained,
D2 optionally ascertaining an equilibrium state by deflecting the transition state, such that a deflected transition state is obtained, and relaxing the deflected transition state by means of the quantum-chemical method, such that an equilibrium state is obtained.

It has been found that, surprisingly, the object can be achieved by using a suitable Monte Carlo method to vary different molecular geometries and approximating a geometry for a transition state with the aid of a quality criterion. Furthermore, it has been found that, surprisingly, the object can be achieved by using the gradient norm of the curve of the direct reaction pathway as an observable in the Monte Carlo method. This allows the function space under consideration, which is to reflect the molecular geometry at the transition state of the chemical reaction, to be reduced to such a degree that the calculation of saddle points is enabled without calculating a potential hypersurface or manually searching for molecular geometries beforehand. Moreover, it has been found that, surprisingly, the method according to the invention enables the calculation of saddle points in function spaces with a high number of dimensions. It is a further advantage of the method according to the invention that the quantum-chemical calculations are performed by the same method in the performance of the method. With current computer technology, it is possible by the method of the invention to much more quickly and rapidly calculate even transition states for molecules having up to 100 atoms, in particular also through the reduction in labour required to implement corresponding conventional processes. There is also a saving here in terms of computer resources.

In the method according to the invention, in step A a starting geometry is first produced by, in step A1, providing the three-dimensional representation of at least one molecule at ground state energy. Then, in step A2, a bond of the molecule represented three-dimensionally in step A1 is selected, and a length of the bond that differs from the length of the bond at ground state energy, such that a starting geometry is obtained. The selected bonds are preferably those bonds which are formed or broken in the chemical reaction in question. The bond length that differs from the length of the bond at ground state energy is 10 to 90%, preferably 20% to 40%, greater than the bond at ground state energy.

Preferably, the at least one molecule from step A1 has a size of not more than 100 atoms, further preferably of not more than 80 atoms, more preferably of not more than 60 atoms, and/or the length of the bond selected in step A2 is not more than 230 pm, preferably not more than 200 pm, further preferably not more than 180 pm, more preferably not more than 150 pm.

In a further preferred embodiment of the method according to the invention, in step A1, at least two molecules I and II are provided and, in step A2, alternatively or additionally to the at least one bond, at least one distance between at least one atom from molecule I and at least one atom from molecule II and the length of the at least one distance may also be selected, where the length of the at least one distance is especially not more than 230 pm, preferably not more than 200 pm, further preferably not more than 180 pm, more preferably not more than 150 pm. When the method is performed with at least two molecules I and II and the length of the distance between atoms of the different molecules is selected, the transition state of a synthesis reaction can preferably be calculated by the method. Preferably, molecule I has a size of ≤100 atoms and molecule II a size of ≤100 atoms; more preferably, molecule I has a size of ≤80 atoms and molecule II a size of ≤80 atoms; even more preferably, molecule I has a size of ≤60 atoms and molecule II a size of ≤60 atoms. Preferably, the sum total of the atoms from molecule I and from molecule II is ≤100 atoms.

Preferably, molecule I is a catalyst for the chemical reaction, especially for a polymer synthesis, and molecule II is a reactant in the chemical reaction. The polymer synthesis in this embodiment is preferably polyurethane syntheses. The chemical reaction in this embodiment is preferably also syntheses of monomers for polymerization reactions, industrially required commodity chemicals, additives, surfactants and active pharmacological ingredients. In particular, the chemical reaction is a synthesis for commodity chemicals or reactants for chemical syntheses that are obtained with catalysts. Additives are generally understood to mean additives for plastics such as plasticizers, antioxidants, modifiers, and additives for fuels, in the synthesis of which catalysts are used in each case.

In step A3, the selected starting geometry obtained in step A2 is represented in Cartesian and/or internal coordinates. Internal coordinates describe the spatial arrangement of the atoms relative to one another using bond lengths, bond angles and torsion angles.

In step B of the method according to the invention, an optimized starting geometry is ascertained. For this purpose, first of all, a function space is defined in step B1. The function space includes the spatial coordinates of selected atoms, where the spatial coordinates in the vector space of all atom coordinates included in the molecule define a subspace. The selected atoms for the function space are a set of atoms that are involved in a bond dissociation or preferably in a synthesis reaction. In addition, the function space includes the at least one bond from step A2. The In the preferred embodiment with at least two molecules I and II, the function space includes the distance between at least one atom from molecule I and at least one atom from molecule II.

In the subsequent step B2, the starting geometry is subjected to a geometry optimization by means of a quantum-chemical method with the boundary condition that the length of the at least one bond selected in step A2 is kept constant, such that an optimized starting geometry is obtained. This geometry optimization includes all the atoms of the molecule selected as starting geometry. In the preferred embodiment with at least two molecules I and II, in the geometry optimization, the distance between at least one atom from molecule I and at least one atom from molecule II is kept constant, such that an optimized starting geometry is obtained.

In the geometry optimization with a boundary condition, the total energy of the molecule is minimized as a function of the nuclear coordinates of the atoms present in the molecule by a quantum-chemical method. In order to obtain a local energy minimum, the energy is minimized by gradients (e.g. steepest descent), with minimization of the energy to such an extent as permitted by the boundary condition.

In step B3, the gradient norm B3 is ascertained for the optimized starting geometry obtained beforehand, where the gradient norm is obtained via the first derivative of a function E=f(x) by means of the quantum-chemical method, with E=total energy of the optimized starting geometry and x=nuclear coordinates of the molecule in the optimized starting geometry. In step B3, the same quantum-chemical method is used as in step B2. The Euclidean norm of this vector is the gradient norm. In other words, the gradient vector of the energy is calculated. The gradient vector defines the same vectorial space as the molecule. Each component of the vector consists of the partial derivative of the energy in the xᵢ coordinate

$$( \frac{E}{E {E}_{E}} E ( E ) )$$

and extends in the direction of the unit vector of the xᵢ coordinate. Thereafter, the (Euclidean) norm of this vector is ascertained.

The further continuation of the method on the size of the gradient norm B3 obtained. If the gradient norm B3 ∇ is 0≤∇≤0.07 Eₕ a₀⁻¹, the optimized starting geometry can be classified as a precursor to the transition state of the chemical reaction and step C of the method omitted and the method continued with step D1. If the gradient norm B3 ∇ is >0.07 Eₕ a₀⁻¹, step C of the method is performed and a precursor to the transition state of the chemical reaction is first ascertained, proceeding from the optimized starting geometry. Eₕ here represents the Hartree energy, where 1 Eₕ=4.3597·10⁻¹⁸ J, and a₀ represents the Bohr radius, where 1 a₀=5.29·10⁻¹¹ m.

In step C, the precursor to the transition state of the chemical reaction is ascertained. For this purpose, the optimized starting geometry is varied using a Monte Carlo algorithm by implementing randomly generated deflections of atoms in an ensemble consisting of one molecule or preferably a geometry of at least two molecules I and II.

Monte Carlo algorithms or methods are simulation methods that analytically solve mathematical problems that are soluble only with difficulty, if at all, by numerical approximations. This is done by describing a most likely progression of an experiment by a multitude of randomly arranged individual experiments.

In the Monte Carlo algorithm according to the invention which is employed in step C, randomly generated deflections of atoms in an ensemble consisting of a molecule or a geometry of at least two molecules I and II are implemented and the change in an observable is observed. This observable is a gradient norm. The method according to the invention here varies only a subspace of distinctly smaller dimensions than the entire function space of the ensemble and considers the change in the observable in the overall ensemble as a function of the reduced function space.

For this purpose, at least one atom is selected in step C1.1 from the function space selected in step B1. This is done with the aid of a random number Z1. The direction of a deflection of the atom chosen by Z1, or the direction of the vector for the deflection, is selected in step C1.2 by a further random number Z2. The direction is preferably defined in that, in the preferred step C1.2a, the position of an atom other than that selected in step C1.1 is selected from the reduced function space and then, in the preferred step C1.2b, the direction of the deflection is selected, where the position of the atom selected in step C1.2a determines the direction of the vector. The amplitude of the deflection is preferably determined via a third random number Z3 in the preferred step C1.2c.

In a preferred embodiment of the method according to the invention, step C1.2 comprises the further following steps of:
C1.2a determining a further atom from the function space selected in step B1 which does not correspond to the atom selected in step C1.1,
C1.2b selecting the direction of the vector, where the position of the atom selected in step C1.2.a determines the direction of the vector,
C1.2.c randomly selecting the length of the vector, where the value of the length of the vector may assume positive or negative values.

By virtue of the position of the atom selected in step C1.2 determining the direction of the vector, the function space is restricted as far as possible, and this course of action also has the effect that primarily bond lengths having lengths as are very probably actually achieved during a reaction are varied.

In step C1.3, the atom selected in step C.1.1 is deflected from its position in the optimized starting geometry using the vector from step C1.2, so as to obtain a precursor to the transition state of the chemical reaction.

The precursor to the transition state obtained is subjected to a geometry optimization in step C2 by means of the quantum-chemical method and with the boundary condition that the at least one bond from step A2 has the length that was ascertained in step C1.3. In step C2, the same quantum-chemical method is used as in steps B2 and B3. Then, in step C3, the gradient norm C3 for the precursor to the transition state from step C2 is ascertained, where the gradient norm is obtained via the first derivative of the function E=f(x) by means of the quantum-chemical method, with E=total energy of the precursor to the transition state and x=nuclear coordinates of the molecule in the precursor to the transition state. In step C3, the same quantum-chemical method is used as in steps B2, B3 and C2.

The further continuation of the method depends on the size of the gradient norm C3 obtained. If the gradient norm C3 ∇ is 0≤∇≤0.07 Eₕ a₀⁻¹, step D1 of the method can be performed with the precursor to the transition state. Eₕ here represents the Hartree energy, where 1 Eₕ=4.3597·10⁻¹⁸ J, and a₀ represents the Bohr radius, where 1 a₀=5.29·10⁻¹¹ m.

If the gradient norm C3 ∇ is >0.07 Eₕ a₀⁻¹, steps C1 to C3 are repeated in step 4.2 until a gradient norm C3 of 0≤∇≤0.07 Eₕ a₀⁻¹ is obtained. The repetition of steps C1 to C3 constitutes an iterative method by which the gradient norm is to be minimized. Therefore, the starting point chosen in each case for the repetition of the method is a molecular geometry that already has a minimum gradient norm. In the repetition of steps C1 to C3, it is thus possible in step C1 to vary not just the optimized starting geometry but also a precursor to the transition state if the value of its gradient norms C3 is less than that of the optimized starting geometry. Preferably, the optimized starting geometry obtained in the performance of the method and precursor(s) to the transition state that are obtained and the values of the respective gradient norms C3 are stored.

If steps C1 to C3 have been performed once, it is possible to choose that molecule geometry with the lowest gradient norm from two molecule geometries to perform the repetition, namely either the optimized starting geometry or the precursor to the transition state obtained in the first run. If steps C1 to C3 have been performed once, the geometry of the precursor to the transition state is varied in step C1 when the value of its gradient norm C3 is lower than the value of the gradient norm B3 of the optimized starting geometry.

If steps C1 to C3 have already been performed, it is possible to choose that molecule geometry with the lowest gradient norm from more than two molecule geometries to perform the repetition, namely either the optimized starting geometry or the (more than one) precursor to the transition state obtained in the repetitions. If steps C1 to C3 have been performed more than once, the geometry of the optimized starting geometry or that precursor to the transition state from the preceding repetitions that has the lowest value for the gradient norm C3 or B3 compared to all the gradient norms C3 and B3 previously obtained is varied in step C1.

Preferably, in step C4.1, the gradient norm C3 is ≤0.05 Eₕ a₀⁻¹, preferably C3≤0.04 Eₕ a₀⁻¹, more preferably ≤0.03 Eₕ a₀⁻¹, and step C4.2 is performed until a gradient norm C3 of ≤0.05 Eₕ a₀⁻¹, preferably C3≤0.04 Eₕ a₀⁻¹, more preferably ≤0.03 Eₕ a₀⁻¹, is obtained.

Preferably, step C4.2 is repeated not more than 50 times, preferably not more than 40 times, more preferably not more than 30 times. Preferably, in the performance of step C4.2, a different atom may be selected in the repetition of step C1 than in the preceding performance of the method.

One advantage of the method according to the invention is that, the in the variation using a Monte Carlo algorithm, only the atom coordinates of the selected function space, i.e. of the subspace of the by the selected atoms involved in the bond dissociation or preferably synthesis reaction in question, is defined in the vector space of all atom coordinates included in the molecule, or in the molecule ensemble. This means that the Monte Carlo algorithm according to the invention operates only in this subspace since its functions are defined only therein. Thus, the subspace of the coordinates involved in the dissociation, or preferably synthesis reaction, is the function space of the Monte Carlo algorithm. The calculation of the energies and the gradient vectors (or norm thereof) depends on all the coordinates in the molecule, but the Monte Carlo algorithm according to the invention considers these only as a function of the coordinates of the subspace. In order that, in this consideration, the function value, i.e. the gradient norm to be minimized, remains clear, each consideration of the function value is preceded by relaxation of the entire molecular structure by a geometry optimization with boundary condition(s). The boundary condition(s) are the bond distances generated by the Monte Carlo algorithm.

In step D1, the precursor from step C.4.1 or the precursor from step B4.1 is relaxed by means of the quantum-chemical method and a pseudo-Newton-Raphson algorithm of a, such that the transition state is obtained. In step D1, the same quantum-chemical method is used as in steps B2, B3, C2 and C3.

For this purpose, preferably, for conversion of the respective molecule geometry to a stationary point (G=0), a geometry optimization is undertaken, in which the total energy is minimized as a function of the nuclear coordinates of the atoms present in the molecule. In order to obtain a local energy minimum, the energy is preferably minimized by gradients (e.g. steepest descent). In order to obtain first-order saddle points (chemically interpretable as transition states), a Newton-Raphson algorithm is used.

In step D2 is optionally ascertaining an equilibrium state by deflecting the transition state, such that a deflected transition state is obtained, and relaxation of the deflected transition state by means of the quantum-chemical method, such that an equilibrium state is obtained. In step D2, the same quantum-chemical method is used as in steps B2, B3, C2, C3 and D1.

The above-described procedure in steps C and D is based on the following fundamental considerations: The transition state is characterized by a saddle point structure of the reaction pathway with a gradient of size zero and a negative curvature along a geometric degree of freedom. If the local energy minima closest to the saddle point structure are to be obtained (chemically interpretable as reactants, products or intermediates), a geometric perturbation of the saddle point structure, i.e. a deflection of the atoms of the molecule, can be implemented, such that a structure with non-vanishing gradients is obtained. If this molecular structure is subjected to a geometry optimization by gradients, it is thus possible to obtain local minima that adjoin the saddle point.

In steps B2, B3, C2, C3, D1 and D2, the same quantum-chemical method is used in each case. Preferably, the quantum-chemical method from steps B2, B3, C2, C3, D1 and D2 is a semiempirical method, density functional theory method or an approximation of the Schrödinger equation, especially preferably density functional theory methods, for example the TPSS density functional with a def2-SVP basis set as implemented in a standard manner in the Turbomole software package. In a preferred embodiment, the quantum-mechanical calculations are conducted with the TURBOMOLE software package. Preference is given to calculating using a computer with 16-core processors with a clock frequency of 3.20 GHz and a 25 MB cache with 128 GB of DDR4 2400 rg ECC RAM.

Preferably, the chemical reaction is a synthesis selected from the group consisting of polymer syntheses, especially polyurethane syntheses, syntheses of monomers for polymerization reactions, industrially required commodity chemicals, additives, surfactants and active pharmacological ingredients. In particular, the chemical reaction is a synthesis for commodity chemicals or reactants for chemical syntheses that are obtained with catalysts. Additives are generally understood to mean additives for plastics such as plasticizers, antioxidants, modifiers, and additives for fuels, in the synthesis of which catalysts are used in each case.

If, in the preferred embodiment of the method, at least two molecules I and II are provided, the steps mentioned under letters A, B and C of the method may preferably be repeated, wherein, in each repetition by comparison with preceding performances of the method,
molecule I is varied or a different molecule is provided as molecule I, and
molecule II is not varied, nor is any other molecule provided as molecule II,
and the additional step D0 may be conducted:
D0 comparing the gradient norm C3 obtained in the repetitions for the various precursors to the transition state and selecting the precursor to the transition state having the lowest gradient norm C3 and performing step D1 and/or D2 with the selected precursor to the transition state.

In this above-described preferred embodiment of the method, the gradient norm C3 is first calculated for different combinations of molecules and the results are stored. Then, in a preferred step D0, the values obtained for the gradient norm C3 may be compared and the combination of molecules with the lowest gradient norm C3 may be selected. This preferred embodiment of the method thus enables direct comparison of different combinations of molecules.

In a further alternatively preferred embodiment, the invention relates to a method wherein information about the transition state ascertained according to step D.1 and/or the equilibrium state ascertained according to step D.2 is communicated to a user.

In a further alternatively preferred embodiment, the invention relates to a method wherein information about the transition state ascertained according to step D.1 and/or the equilibrium state ascertained according to step D.2 is received by a user.

In a further alternatively preferred embodiment, the invention relates to a method wherein the molecule I is synthesized after step D.1 and/or after step D.2.

In a further alternatively preferred embodiment, the invention relates to a method wherein under a chemical reaction with the molecule I as catalyst is performed after step D.1 and/or after step D.2.

In a further alternatively preferred embodiment, the invention relates to a method wherein under a chemical reaction with the molecule II as co-reactant is performed after step D.1 and/or after step D.2.

The invention further provides a system for data processing, comprising means of executing a method according to the invention.

The invention further provides a computer program comprising commands that, on execution of the program by a computer, cause it to perform the following steps of a method:

A Generating a Starting Geometry
A1 providing the three-dimensional representation of at least one molecule at ground state energy,
A2 selecting at least one bond of the at least one molecule and selecting a length of the bond, where the selected length does not correspond to the length of the bond at ground state energy of the molecule, such that a starting geometry for the chemical reaction is obtained,
A3 representing the starting geometry in three dimensions in Cartesian and/or internal coordinates,

B Ascertaining an Optimized Starting Geometry
B1 defining a function space encompassing the at least one bond from step A2 and the atoms joined by this bond,
B2 optimizing the geometry of the starting geometry on the basis of the function space selected in step B1 by means of a quantum-chemical method and with the boundary condition that the length of the at least one bond selected in step A2 is kept constant, such that an optimized starting geometry is obtained,
B3 ascertaining the gradient norm B3 for the optimized starting geometry, where the gradient norm is obtained via the first derivative of a function E=f(x) by means of the quantum-chemical method, with E=total energy of the optimized starting geometry and x=nuclear coordinates of the molecule in the optimized starting geometry,
B4.1 when the gradient norm B3 ∇ is 0≤∇≤0.07 Eₕ a₀⁻¹, classifying the optimized starting geometry as a precursor to the transition state of the chemical reaction and continuing the method with step D1, or
B4.2 when the gradient norm B3 ∇ is >0.07 Eₕ a₀⁻¹, ascertaining the precursor to the transition state of the chemical reaction proceeding from the optimized starting geometry by a method comprising the following steps:

C Ascertaining the Precursor to the Transition State of the Chemical Reaction
C1 varying the optimized starting geometry using a Monte Carlo algorithm, wherein
C1.1 at least one atom from the function space selected in step B1 is selected at random,
C1.2 a vector for a deflection of the atom chosen in step C1.1 is selected at random,
C1.3 the atom selected in step C1.1 is deflected from its position in the optimized starting geometry using the vector from step C1.2, so as to obtain a precursor to the transition state of the chemical reaction,
C2 optimizing the geometry of the precursor to the transition state by means of the quantum-chemical method and with the boundary condition that the at least one bond from step A2 has the length that was ascertained in step C1.3,
C3 ascertaining the gradient norm C3 for the precursor to the transition state from step C2, where the gradient norm is obtained via the first derivative of the function E=f(x) by means of the quantum-chemical method, with E=total energy of the precursor to the transition state and x=nuclear coordinates of the molecule in the precursor to the transition state,
C4.1 when the gradient norm C3 ∇ is 0≤∇≤0.07 Eₕ a₀⁻¹, continuing the method with step D1, or
C4.2 when the gradient norm C3 ∇ is >0.07 Eₕ a₀⁻¹, repeating steps C1 to C3 until a gradient norm C3 of 0≤∇≤0.07 Eₕ a₀⁻¹ is obtained, wherein
(a) if steps C1 to C3 have been performed once, the geometry of the precursor to the transition state is varied in step C1 when the value of its gradient norm C3 is lower than the value of the gradient norm B3 of the optimized starting geometry, or
(b) if steps C1 to C3 have been performed more than once, the geometry of the optimized starting geometry or that precursor to the transition state from the preceding repetitions that has the lowest value for the gradient norm C3 or B3 compared to all the gradient norms C3 and B3 previously obtained is varied in step C1,

D Ascertaining the Transition State
D1 relaxing the precursor from step C4.1 or the precursor from step B4.1 by means of the quantum-chemical method and a pseudo-Newton-Raphson algorithm, such that the transition state is obtained,
D2 optionally ascertaining an equilibrium state by deflecting the transition state, such that a deflected transition state is obtained, and relaxing the deflected transition state by means of the quantum-chemical method, such that an equilibrium state is obtained.

Preferably, the computer program comprises commands that, on execution of the program by a computer, cause it to perform steps B to D of the method.

The invention also provides a computer-readable storage medium comprising commands that, on execution by a computer, cause it to perform the following steps of a method:

A Generating a Starting Geometry
A1 providing the three-dimensional representation of at least one molecule at ground state energy,
A2 selecting at least one bond of the at least one molecule and selecting a length of the bond, where the selected length does not correspond to the length of the bond at ground state energy of the molecule, such that a starting geometry for the chemical reaction is obtained,
A3 representing the starting geometry in three dimensions in Cartesian and/or internal coordinates,

B Ascertaining an Optimized Starting Geometry
B1 defining a function space encompassing the at least one bond from step A2 and the atoms joined by this bond,
B2 optimizing the geometry of the starting geometry on the basis of the function space selected in step B1 by means of a quantum-chemical method and with the boundary condition that the length of the at least one bond selected in step A2 is kept constant, such that an optimized starting geometry is obtained,
B3 ascertaining the gradient norm B3 for the optimized starting geometry, where the gradient norm is obtained via the first derivative of a function E=f(x) by means of the quantum-chemical method, with E=total energy of the optimized starting geometry and x=nuclear coordinates of the molecule in the optimized starting geometry,
B4.1 when the gradient norm B3 ∇ is 0≤∇≤0.07 Eₕ a₀⁻¹, classifying the optimized starting geometry as a precursor to the transition state of the chemical reaction and continuing the method with step D1, or
B4.2 when the gradient norm B3 ∇ is >0.07 Eₕ a₀⁻¹, ascertaining the precursor to the transition state of the chemical reaction proceeding from the optimized starting geometry by a method comprising the following steps:

C Ascertaining the Precursor to the Transition State of the Chemical Reaction
C1 varying the optimized starting geometry using a Monte Carlo algorithm, wherein
C1.1 at least one atom from the function space selected in step B1 is selected at random,
C1.2 a vector for a deflection of the atom chosen in step C1.1 is selected at random,
C1.3 the atom selected in step C1.1 is deflected from its position in the optimized starting geometry using the vector from step C1.2, so as to obtain a precursor to the transition state of the chemical reaction,
C2 optimizing the geometry of the precursor to the transition state by means of the quantum-chemical method and with the boundary condition that the at least one bond from step A2 has the length that was ascertained in step C1.3,
C3 ascertaining the gradient norm C3 for the precursor to the transition state from step C2, where the gradient norm is obtained via the first derivative of the function E=f(x) by means of the quantum-chemical method, with E=total energy of the precursor to the transition state and x=nuclear coordinates of the molecule in the precursor to the transition state,
C4.1 when the gradient norm C3 ∇ is 0≤∇≤0.07 Eₕ a₀⁻¹, continuing the method with step D1, or
C4.2 when the gradient norm C3 ∇ is >0.07 Eₕ a₀⁻¹, repeating steps C1 to C3 until a gradient norm C3 of 0≤∇≤0.07 Eₕ a₀⁻¹ is obtained, wherein
(a) if steps C1 to C3 have been performed once, the geometry of the precursor to the transition state is varied in step C1 when the value of its gradient norm C3 is lower than the value of the gradient norm B3 of the optimized starting geometry, or
(b) if steps C1 to C3 have been performed more than once, the geometry of the optimized starting geometry or that precursor to the transition state from the preceding repetitions that has the lowest value for the gradient norm C3 or B3 compared to all the gradient norms C3 and B3 previously obtained is varied in step C1,

D Ascertaining the Transition State
D1 relaxing the precursor from step C4.1 or the precursor from step B4.1 by means of the quantum-chemical method and a pseudo-Newton-Raphson algorithm, such that the transition state is obtained,
D2 optionally ascertaining an equilibrium state by deflecting the transition state, such that a deflected transition state is obtained, and relaxing the deflected transition state by means of the quantum-chemical method, such that an equilibrium state is obtained.

Preferably, the computer-readable storage medium comprises commands that, on execution of the program by a computer, cause it to perform steps B to D of the method. The computer-readable storage medium here may be, for example, one or more physical hard disk(s) suitable for storing programs for executing commands.

The invention further relates to the use of the computer program according to the invention or of the computer-readable storage medium according to the invention for assessing transition states of a chemical reaction, especially a polymer synthesis.

The invention especially relates to the following embodiments:

In a first embodiment, the invention relates to a computer-implemented method of calculating transition states of a chemical reaction, comprising the steps of

A Generating a Starting Geometry
A1 providing the three-dimensional representation of at least one molecule at ground state energy,
A2 selecting at least one bond of the at least one molecule and selecting a length of the bond, where the selected length does not correspond to the length of the bond at ground state energy of the molecule, such that a starting geometry for the chemical reaction is obtained,
A3 representing the starting geometry in three dimensions in Cartesian and/or internal coordinates,

B Ascertaining an Optimized Starting Geometry
B1 defining a function space encompassing the at least one bond from step A2 and the atoms joined by this bond,
B2 optimizing the geometry of the starting geometry on the basis of the function space selected in step B1 by means of a quantum-chemical method and with the boundary condition that the length of the at least one bond selected in step A2 is kept constant, such that an optimized starting geometry is obtained,
B3 ascertaining the gradient norm B3 for the optimized starting geometry, where the gradient norm is obtained via the first derivative of a function E=f(x) by means of the quantum-chemical method, with E=total energy of the optimized starting geometry and x=nuclear coordinates of the molecule in the optimized starting geometry,
B4.1 when the gradient norm B3 ∇ is 0≤∇≤0.07 Eₕ a₀⁻¹, classifying the optimized starting geometry as a precursor to the transition state of the chemical reaction and continuing the method with step D1, or
B4.2 when the gradient norm B3 ∇ is >0.07 Eₕ a₀⁻¹, ascertaining the precursor to the transition state of the chemical reaction proceeding from the optimized starting geometry by a method comprising the following steps:

C Ascertaining the Precursor to the Transition State of the Chemical Reaction
C1 varying the optimized starting geometry using a Monte Carlo algorithm, wherein
C1.1 at least one atom from the function space selected in step B1 is selected at random,
C1.2 a vector for a deflection of the atom chosen in step C1.1 is selected at random,
C1.3 the atom selected in step C1.1 is deflected from its position in the optimized starting geometry using the vector from step C1.2, so as to obtain a precursor to the transition state of the chemical reaction,
C2 optimizing the geometry of the precursor to the transition state by means of the quantum-chemical method and with the boundary condition that the at least one bond from step A2 has the length that was ascertained in step C1.3,
C3 ascertaining the gradient norm C3 for the precursor to the transition state from step C2, where the gradient norm is obtained via the first derivative of the function E=f(x) by means of the quantum-chemical method, with E=total energy of the precursor to the transition state and x=nuclear coordinates of the molecule in the precursor to the transition state,
C4.1 when the gradient norm C3 ∇ is 0≤∇≤0.07 Eₕ a₀⁻¹, continuing the method with step D1, or
C4.2 when the gradient norm C3 ∇ is >0.07 Eₕ a₀⁻¹, repeating steps C1 to C3 until a gradient norm C3 of 0≤∇≤0.07 Eₕ a₀⁻¹ is obtained, wherein
(a) if steps C1 to C3 have been performed once, the geometry of the precursor to the transition state is varied in step C1 when the value of its gradient norm C3 is lower than the value of the gradient norm B3 of the optimized starting geometry, or
(b) if steps C1 to C3 have been performed more than once, the geometry of the optimized starting geometry or that precursor to the transition state from the preceding repetitions that has the lowest value for the gradient norm C3 or B3 compared to all the gradient norms C3 and B3 previously obtained is varied in step C1,

D Ascertaining the Transition State
D1 relaxing the precursor from step C4.1 or the precursor from step B4.1 by means of the quantum-chemical method and a pseudo-Newton-Raphson algorithm, such that the transition state is obtained,
D2 optionally ascertaining an equilibrium state by deflecting the transition state, such that a deflected transition state is obtained, and relaxing the deflected transition state by means of the quantum-chemical method, such that an equilibrium state is obtained.

In a second embodiment, the invention relates to a method according to Embodiment 1, characterized in that the at least one molecule from step A1 has a size of not more than 100 atoms and/or in that the length of the bond selected in step A2 is not more than 230 pm.

In a third embodiment, the invention relates to a method according to Embodiment 1 or 2, characterized in that step C1.2 comprises the further following steps:
C1.2a determining a further atom from the function space selected in step B1 which does not correspond to the atom selected in step C1.1,
C1.2b selecting the direction of the vector, where the position of the atom selected in step C1.2.a determines the direction of the vector,
C1.2.c randomly selecting the length of the vector, where the value of the length of the vector may assume positive or negative values.

In a fourth embodiment, the invention relates to a method according to any of the above embodiments, characterized in that, in step C4.1, the gradient norm C3 is ≤0.05 Eₕ a₀⁻¹, preferably C3≤0.04 Eₕ a₀⁻¹, more preferably ≤0.03 Eₕ a₀⁻¹, and step C4.2 is performed until a gradient norm C3≤0.05 Eₕ a₀⁻¹, preferably C3≤0.04 Eₕ a₀⁻¹, more preferably ≤0.03 Eₕ a₀⁻¹, is obtained.

In a fifth embodiment, the invention relates to a method according to any of the preceding embodiments, characterized in that step C4.2 is repeated not more than 50 times, preferably not more than 30 times.

In a sixth embodiment, the invention relates to a method according to any of the preceding embodiments, characterized in that, in the performance of step C4.2, a different atom may be selected in the repetition of step C1 than in the preceding performance of the method.

In a seventh embodiment, the invention relates to a method according to any of the preceding embodiments, characterized in that the quantum-chemical method from steps B2, B3, C2, C3, D1 and D2 is a semiempirical method, density functional theory method or an approximation of the Schrödinger equation, the quantum-chemical method from steps B2, B3, C2, C3, D1 and D2 especially being a density functional theory method.

In an eighth embodiment, the invention relates to a method according to any of the preceding embodiments, characterized in that the chemical reaction is a synthesis selected from the group consisting of polymer syntheses, especially polyurethane syntheses, syntheses of monomers for polymerization reactions, industrially required commodity chemicals, additives, surfactants and active pharmacological ingredients.

In a ninth embodiment, the invention relates to a method according to any of the preceding embodiments, characterized in that, in step A1, at least two molecules I and II are provided and, in step A2, alternatively or additionally to the at least one bond, at least one distance between at least one atom from molecule I and at least one atom from molecule II and the length of the at least one distance may also be selected, where the length of the distance is especially not more than 230 pm.

In a tenth embodiment, the invention relates to a method according to Embodiment 9, characterized in that the steps mentioned under letters A, B and C of the method are repeated, wherein, in each repetition by comparison with preceding performances of the method,
molecule I is varied or a different molecule is provided as molecule I, and
molecule II is not varied, nor is any other molecule provided as molecule II,
and comprising an additional step of
D0 comparing the gradient norm C3 obtained in the repetitions for the various precursors to the transition state and selecting the precursor to the transition state having the lowest gradient norm C3 and performing step D1 and/or D2 with the selected precursor to the transition state.

In an eleventh embodiment, the invention relates to a method according to either of Embodiments 9 and 10, characterized in that molecule I is a catalyst for the chemical reaction, especially for a polymer synthesis, and molecule II is a reactant in the chemical reaction.

In a twelfth embodiment, the invention relates to a method according to any of Embodiments 9 to 11, characterized in that the length of the distance between at least one atom from molecule I and/or at least one atom from molecule II that has been selected in step A2 is not more than 230 pm.

In a thirteenth embodiment, the invention relates to a method according to any of Embodiments 9 to 12, characterized in that molecule I has a size of ≤100 atoms and molecule II has a size of ≤100 atoms, and the sum total of the atoms from molecule I and from molecule II should preferably be ≤100 atoms.

In a fourteenth embodiment, the invention relates to a system for data processing, comprising means of executing a method comprising the steps of:

A Generating a Starting Geometry
A1 providing the three-dimensional representation of at least one molecule at ground state energy,
A2 selecting at least one bond of the at least one molecule and selecting a length of the bond, where the selected length does not correspond to the length of the bond at ground state energy of the molecule, such that a starting geometry for the chemical reaction is obtained,
A3 representing the starting geometry in three dimensions in Cartesian and/or internal coordinates,

B Ascertaining an Optimized Starting Geometry
B1 defining a function space encompassing the at least one bond from step A2 and the atoms joined by this bond,
B2 optimizing the geometry of the starting geometry on the basis of the function space selected in step B1 by means of a quantum-chemical method and with the boundary condition that the length of the at least one bond selected in step A2 is kept constant, such that an optimized starting geometry is obtained,
B3 ascertaining the gradient norm B3 for the optimized starting geometry, where the gradient norm is obtained via the first derivative of a function E=f(x) by means of the quantum-chemical method, with E=total energy of the optimized starting geometry and x=nuclear coordinates of the molecule in the optimized starting geometry,
B4.1 when the gradient norm B3 ∇ is 0≤∇≤0.07 Eₕ a₀⁻¹, classifying the optimized starting geometry as a precursor to the transition state of the chemical reaction and continuing the method with step D1, or
B4.2 when the gradient norm B3 ∇ is >0.07 Eₕ a₀⁻¹, ascertaining the precursor to the transition state of the chemical reaction proceeding from the optimized starting geometry by a method comprising the following steps:

C Ascertaining the Precursor to the Transition State of the Chemical Reaction
C1 varying the optimized starting geometry using a Monte Carlo algorithm, wherein
C1.1 at least one atom from the function space selected in step B1 is selected at random,
C1.2 a vector for a deflection of the atom chosen in step C1.1 is selected at random,
C1.3 the atom selected in step C1.1 is deflected from its position in the optimized starting geometry using the vector from step C1.2, so as to obtain a precursor to the transition state of the chemical reaction,
C2 optimizing the geometry of the precursor to the transition state by means of the quantum-chemical method and with the boundary condition that the at least one bond from step A2 has the length that was ascertained in step C1.3,
C3 ascertaining the gradient norm C3 for the precursor to the transition state from step C2, where the gradient norm is obtained via the first derivative of the function E=f(x) by means of the quantum-chemical method, with E=total energy of the precursor to the transition state and x=nuclear coordinates of the molecule in the precursor to the transition state,
C4.1 when the gradient norm C3 ∇ is 0≤∇≤0.07 Eₕ a₀⁻¹, continuing the method with step D1, or
C4.2 when the gradient norm C3 ∇ is >0.07 Eₕ a₀⁻¹, repeating steps C1 to C3 until a gradient norm C3 of 0≤∇≤0.07 Eₕ a₀⁻¹ is obtained, wherein
(a) if steps C1 to C3 have been performed once, the geometry of the precursor to the transition state is varied in step C1 when the value of its gradient norm C3 is lower than the value of the gradient norm B3 of the optimized starting geometry, or
(b) if steps C1 to C3 have been performed more than once, the geometry of the optimized starting geometry or that precursor to the transition state from the preceding repetitions that has the lowest value for the gradient norm C3 or B3 compared to all the gradient norms C3 and B3 previously obtained is varied in step C1,

D Ascertaining the Transition State
D1 relaxing the precursor from step C4.1 or the precursor from step B4.1 by means of the quantum-chemical method and a pseudo-Newton-Raphson algorithm, such that the transition state is obtained,
D2 optionally ascertaining an equilibrium state by deflecting the transition state, such that a deflected transition state is obtained, and relaxing the deflected transition state by means of the quantum-chemical method, such that an equilibrium state is obtained.

In a fifteenth embodiment, the invention relates to a computer program comprising commands that, on execution of the program by a computer, cause it to perform the following steps of a method:

A Generating a Starting Geometry
A1 providing the three-dimensional representation of at least one molecule at ground state energy,
A2 selecting at least one bond of the at least one molecule and selecting a length of the bond, where the selected length does not correspond to the length of the bond at ground state energy of the molecule, such that a starting geometry for the chemical reaction is obtained,
A3 representing the starting geometry in three dimensions in Cartesian and/or internal coordinates,

B Ascertaining an Optimized Starting Geometry
B1 defining a function space encompassing the at least one bond from step A2 and the atoms joined by this bond,
B2 optimizing the geometry of the starting geometry on the basis of the function space selected in step B1 by means of a quantum-chemical method and with the boundary condition that the length of the at least one bond selected in step A2 is kept constant, such that an optimized starting geometry is obtained,
B3 ascertaining the gradient norm B3 for the optimized starting geometry, where the gradient norm is obtained via the first derivative of a function E=f(x) by means of the quantum-chemical method, with E=total energy of the optimized starting geometry and x=nuclear coordinates of the molecule in the optimized starting geometry,
B4.1 when the gradient norm B3 ∇ is 0≤∇≤0.07 Eₕ a₀⁻¹, classifying the optimized starting geometry as a precursor to the transition state of the chemical reaction and continuing the method with step D1, or
B4.2 when the gradient norm B3 ∇ is >0.07 Eₕ a₀⁻¹, ascertaining the precursor to the transition state of the chemical reaction proceeding from the optimized starting geometry by a method comprising the following steps:

C Ascertaining the Precursor to the Transition State of the Chemical Reaction
C1 varying the optimized starting geometry using a Monte Carlo algorithm, wherein
C1.1 at least one atom from the function space selected in step B1 is selected at random,
C1.2 a vector for a deflection of the atom chosen in step C1.1 is selected at random,
C1.3 the atom selected in step C1.1 is deflected from its position in the optimized starting geometry using the vector from step C1.2, so as to obtain a precursor to the transition state of the chemical reaction,
C2 optimizing the geometry of the precursor to the transition state by means of the quantum-chemical method and with the boundary condition that the at least one bond from step A2 has the length that was ascertained in step C1.3,
C3 ascertaining the gradient norm C3 for the precursor to the transition state from step C2, where the gradient norm is obtained via the first derivative of the function E=f(x) by means of the quantum-chemical method, with E=total energy of the precursor to the transition state and x=nuclear coordinates of the molecule in the precursor to the transition state,
C4.1 when the gradient norm C3 ∇ is 0≤∇≤0.07 Eₕ a₀⁻¹, continuing the method with step D1, or
C4.2 when the gradient norm C3 ∇ is >0.07 Eₕ a₀⁻¹, repeating steps C1 to C3 until a gradient norm C3 of 0≤∇≤0.07 Eₕ a₀⁻¹ is obtained, wherein
(a) if steps C1 to C3 have been performed once, the geometry of the precursor to the transition state is varied in step C1 when the value of its gradient norm C3 is lower than the value of the gradient norm B3 of the optimized starting geometry, or
(b) if steps C1 to C3 have been performed more than once, the geometry of the optimized starting geometry or that precursor to the transition state from the preceding repetitions that has the lowest value for the gradient norm C3 or B3 compared to all the gradient norms C3 and B3 previously obtained is varied in step C1,

D Ascertaining the Transition State
D1 relaxing the precursor from step C4.1 or the precursor from step B4.1 by means of the quantum-chemical method and a pseudo-Newton-Raphson algorithm, such that the transition state is obtained,
D2 optionally ascertaining an equilibrium state by deflecting the transition state, such that a deflected transition state is obtained, and relaxing the deflected transition state by means of the quantum-chemical method, such that an equilibrium state is obtained.

In a sixteenth embodiment, the invention relates to a computer-readable storage medium comprising commands that, on execution by a computer, cause it to perform the following steps of a method:

A Generating a Starting Geometry
A1 providing the three-dimensional representation of at least one molecule at ground state energy,
A2 selecting at least one bond of the at least one molecule and selecting a length of the bond, where the selected length does not correspond to the length of the bond at ground state energy of the molecule, such that a starting geometry for the chemical reaction is obtained,
A3 representing the starting geometry in three dimensions in Cartesian and/or internal coordinates,

B Ascertaining an Optimized Starting Geometry
B1 defining a function space encompassing the at least one bond from step A2 and the atoms joined by this bond,
B2 optimizing the geometry of the starting geometry on the basis of the function space selected in step B1 by means of a quantum-chemical method and with the boundary condition that the length of the at least one bond selected in step A2 is kept constant, such that an optimized starting geometry is obtained,
B3 ascertaining the gradient norm B3 for the optimized starting geometry, where the gradient norm is obtained via the first derivative of a function E=f(x) by means of the quantum-chemical method, with E=total energy of the optimized starting geometry and x=nuclear coordinates of the molecule in the optimized starting geometry,
B4.1 when the gradient norm B3 ∇ is 0≤∇≤0.07 Eₕ a₀⁻¹, classifying the optimized starting geometry as a precursor to the transition state of the chemical reaction and continuing the method with step D1, or
B4.2 when the gradient norm B3 ∇ is >0.07 Eₕ a₀⁻¹, ascertaining the precursor to the transition state of the chemical reaction proceeding from the optimized starting geometry by a method comprising the following steps:

C Ascertaining the Precursor to the Transition State of the Chemical Reaction
C1 varying the optimized starting geometry using a Monte Carlo algorithm, wherein
C1.1 at least one atom from the function space selected in step B1 is selected at random,
C1.2 a vector for a deflection of the atom chosen in step C1.1 is selected at random,
C1.3 the atom selected in step C1.1 is deflected from its position in the optimized starting geometry using the vector from step C1.2, so as to obtain a precursor to the transition state of the chemical reaction,
C2 optimizing the geometry of the precursor to the transition state by means of the quantum-chemical method and with the boundary condition that the at least one bond from step A2 has the length that was ascertained in step C1.3,
C3 ascertaining the gradient norm C3 for the precursor to the transition state from step C2, where the gradient norm is obtained via the first derivative of the function E=f(x) by means of the quantum-chemical method, with E=total energy of the precursor to the transition state and x=nuclear coordinates of the molecule in the precursor to the transition state,
C4.1 when the gradient norm C3 ∇ is 0≤∇≤0.07 Eₕ a₀⁻¹, continuing the method with step D1, or
C4.2 when the gradient norm C3 ∇ is >0.07 Eₕ a₀⁻¹, repeating steps C1 to C3 until a gradient norm C3 of 0≤∇≤0.07 Eₕ a₀⁻¹ is obtained, wherein
(a) if steps C1 to C3 have been performed once, the geometry of the precursor to the transition state is varied in step C1 when the value of its gradient norm C3 is lower than the value of the gradient norm B3 of the optimized starting geometry, or
(b) if steps C1 to C3 have been performed more than once, the geometry of the optimized starting geometry or that precursor to the transition state from the preceding repetitions that has the lowest value for the gradient norm C3 or B3 compared to all the gradient norms C3 and B3 previously obtained is varied in step C1,

D Ascertaining the Transition State
D1 relaxing the precursor from step C4.1 or the precursor from step B4.1 by means of the quantum-chemical method and a pseudo-Newton-Raphson algorithm, such that the transition state is obtained,
D2 optionally ascertaining an equilibrium state by deflecting the transition state, such that a deflected transition state is obtained, and relaxing the deflected transition state by means of the quantum-chemical method, such that an equilibrium state is obtained.

In a seventeenth embodiment, the invention relates to the use of a computer program according to Embodiment 15 or of a computer-readable storage medium according to Embodiment 18 for evaluating transition states of a chemical reaction, especially a polymer synthesis.

In an eighteenth embodiment, the invention relates to methods according to any of the first to thirteenth embodiments, characterized in that information about the transition state ascertained according to step D.1 and/or the equilibrium state ascertained according to step D.2 is communicated to a user.

In a nineteenth embodiment, the invention relates to methods according to any of the first to thirteenth embodiments, characterized in that information about the transition state ascertained according to step D.1 and/or the equilibrium state ascertained according to step D.2 is received by a user.

In a twentieth embodiment, the invention relates to methods according to any of the ninth to thirteenth embodiments, characterized in that the molecule I is synthesized after step D.1 and/or after step D.2.

In a twenty-first embodiment, the invention relates to methods according to any of the ninth to thirteenth embodiments, characterized in that under a chemical reaction with the molecule I as catalyst is performed after step D.1 and/or after step D.2.

In a twenty-second embodiment, the invention relates to methods according to any of the ninth to thirteenth or twenty-first embodiments, characterized in that under a chemical reaction with the molecule II as co-reactant is performed after step D.1 and/or after step D.2.

The invention is to be illustrated by the examples that follow, but without being limited thereto.

### A EXAMPLES FOR ASCERTAINING A PRE-OPTIMIZED STARTING GEOMETRY

The method according to the invention and a conventional method were applied to various reactions. For this purpose, first of all, for each reaction, with the aid of a commercially available program for generating and visualizing three-dimensional structures of chemical molecules, a starting geometry of the unknown transition state sought was drawn. This involved estimating the distances between the atoms or bonds by extending the typical bond lengths in the ground state by 10-90%.

In the case of the working example shown in Table A2, for example, in method step A1, an ethene molecule was drawn together with a butadiene molecule, by means of a molecular visualization program, for example TMoleX or Avogadro. In method step A2, the two bond axes of the terminal double-bonded carbons leading to the cyclohexene were adjusted to a distance of 179.7 pm and 164.1 pm, and the molecular geometry thus generated, in method step A3, was stored in a representation of Cartesian coordinates, by means of a molecular visualization program, for example TMoleX or Avogadro. The atoms involved in the bond distances in such an arrangement were then used to define the function space according to method step B1.

In comparative experiments, the starting geometry was subjected to a geometry optimization by a quasi-Newton-Raphson method as used in the prior art. For this purpose, first of all, the bond distances involved in the bond dissociation were kept constant and a pre-optimization was conducted. Subsequently, the quasi-Newton-Raphson method was employed. It was not possible here to localize any transition state. A repetition of the last step does not bring any change in the result since it is a deterministic method.

In the experiments according to the invention, the method according to the invention was applied to the same starting geometry. For this purpose, first of all, the pre-optimization was implemented by the Monte Carlo method according to the invention. The Monte Carlo method according to the invention varies the fixed bond lengths having dissociation character by random numbers and subsequently performs geometry optimizations with bond distances kept constant. This procedure is run iteratively until the gradient norm obtained is minimized below a fixed value, which indicates a sufficient proximity to a stationary point. For every reaction examined, the Monte Carlo method according to the invention chose 10 optimized starting geometries each with the same value for the gradient norm as the starting point for the subsequent geometry optimization by means of quasi-Newton-Raphson.

The calculations and the success rates, i.e. the number of transition states that could be ascertained on a basis of ten optimized starting geometries in each case, are shown in Tables A1 to A5.

All quantum-mechanical calculations were conducted with the TURBOMOLE software package. The density functional theory method used was the TPSS density functional with a def2-SVP basis set, as implemented as standard in the Turbomole software package. For this purpose, the computer was an Intel Xeon E5-2667v4 with 16-core processors with a clock frequency of 3.20 GHz and a 25 MB cache with 128 GB of DDR4 2400 rg ECC RAM.

Using this computer, it was possible by the method according to the invention, within 2 hours at most, to calculate different transition states for a reaction for 10 different manually selected starting geometries thereof. It was possible here for the method to be performed by the computer even without monitoring by an operator, for example overnight.

By conventional methods in which the starting geometry is varied manually, i.e. without a computer-implemented method, a time of 2 to 3 days per starting geometry would be required until a geometry to which a pseudo-Newton-Raphson method can be applied had been found; in other words, only after 2 to 3 would it possible to even try to calculate a transition state.

Table A.1 below gives a summary of how often step B, or steps B and C, were performed
for all optimized starting geometries for each reaction examined before a precursor to the transition state was obtained or the method was stopped because no precursor to the transition state had been obtained (line 1), or
for those optimized starting geometries for which a transition state could be ascertained (line 2).

TABLE A.1
Statistical evaluation

| Line | Examples | Tab. 2 | Tab. 3 | Tab. 4 | Tab. 5 |
|---|---|---|---|---|---|
| 1 | Average number of runs of step C of | 6.6 | 9.7 | 8.2 | 9.2 |
| | the method according to the invention | | | | |
| | for all optimized starting geometries | | | | |
| | of a reaction | | | | |
| 2 | Average number of runs of step C of | 5.5 | 12.3 | 8.2 | 9.2 |
| | the method according to the invention | | | | |
| | for those optimized starting geometries | | | | |
| | for which a transition state could be | | | | |
| | ascertained | | | | |
| 3 | Percentage of the respective optimized | 80 | 30 | 100 | 100 |
| | starting geometries of an experiment | | | | |
| | for which the method according to the | | | | |
| | invention was able to ascertain a | | | | |
| | transition state | | | | |

For the bromination of ethene, as shown in Table A3 below, it is relatively difficult to calculate a transition state. This is very probably because the saddle point has a very pointed shape.

TABLE A.2
Cycloaddition of 1,3-butadiene and ethene

| | Each column shows a different optimized starting geometry | | | | | | | | | |
|---|---|---|---|---|---|---|---|---|---|---|
| Example | 2.1 | 2.2 | 2.3 | 2.4 | 2.5 | 2.6 | 2.7 | 2.8 | 2.9 | 2.10 |
| Gradient norm | 0.101578 | 0.101578 | 0.101578 | 0.101578 | 0.101578 | 0.101578 | 0.101578 | 0.101578 | 0.101578 | 0.101578 |
| B3 [Eₕ a₀⁻¹] of | | | | | | | | | | |
| the respective | | | | | | | | | | |
| optimized | | | | | | | | | | |
| starting geometry | | | | | | | | | | |
| Gradient norm | 0.063475 | 0.112442 | 0.027745 | 0.094052 | 0.125426 | 0.086226 | 0.081093 | 0.107781 | 0.087146 | 0.125814 |
| C3 [Eₕ a₀⁻¹], | 0.074802 | 0.074597 | | 0.029719 | 0.091124 | 0.088933 | 0.095923 | 0.111804 | 0.159795 | 0.041008 |
| where each line | 0.357504 | 0.090029 | | | 0.065701 | 0.009156 | 0.206321 | 0.070290 | 0.110302 | 0.667969 |
| represents the | 0.090818 | 0.390142 | | | 0.053928 | | 0.063935 | 0.072940 | 0.118519 | 0.194443 |
| gradient norm | 0.111428 | 0.092513 | | | 0.012537 | | 0.012706 | 0.056430 | 2,170,497 | 0.013739 |
| of a different | 1,404,968 | 0.067496 | | | | | | 0.061099 | 0.071430 | |
| precursor to the | 0.111726 | 0.029182 | | | | | | 0.025372 | 0.085191 | |
| transition state, | 0.102065 | | | | | | | | 0.024142 | |
| based in each | 0.077888 | | | | | | | | | |
| case on the | 0.085817 | | | | | | | | | |
| optimized starting | 0.057883 | | | | | | | | | |
| geometry of | 0.194193 | | | | | | | | | |
| the first line | 0.037484 | | | | | | | | | |
| Is a transition | No | No | Yes | Yes | Yes | Yes | Yes | Yes | Yes | Yes |
| state obtained? | | | | | | | | | | |

In Example 2.1, the repetition of step C of the method according to the invention was stopped after 13 runs because it was not possible to ascertain a to the transition state that was suitable as a starting point for the ascertaining of a transition state with the aid of a pseudo-Newton-Raphson precursor algorithm according to step DI.

TABLE A.3
Bromination of ethene to give 1,2-bromoethane

| | Each column shows a different optimized starting geometry | | | | | | | | | |
|---|---|---|---|---|---|---|---|---|---|---|
| Example | 3.1 | 3.2 | 3.3 | 3.4 | 3.5 | 3.6 | 3.7 | 3.8 | 3.9 | 3.10 |
| Gradient norm | 0.056813 | 0.056813 | 0.056813 | 0.056813 | 0.056813 | 0.056813 | 0.056813 | 0.056813 | 0.056813 | 0.056813 |
| B3 [Eₕ a₀⁻¹] of | | | | | | | | | | |
| the respective | | | | | | | | | | |
| optimized | | | | | | | | | | |
| starting geometry | | | | | | | | | | |
| Gradient norm | 0.058529 | 0.064647 | 0.064082 | 0.048314 | 0.031738 | 0.058399 | 0.051681 | 0.058077 | 0.127864 | 0.231416 |
| C3 [Eₕ a₀⁻¹], | 0.251325 | 0.061090 | 0.152230 | 0.137252 | | 0.059887 | 0.044425 | 0.060593 | 0.232556 | 0.056812 |
| where each line | 0.064038 | 0.107419 | 0.051544 | 0.056970 | | 0.060656 | 0.066049 | 0.053602 | 0.057414 | 0.063343 |
| represents the | 0.056935 | 0.065046 | 0.099173 | 0.048362 | | 0.284270 | 0.081247 | 0.070952 | 0.044737 | 0.065012 |
| gradient norm | 0.059306 | 0.062879 | 0.112987 | 0.056837 | | 0.058757 | 0.057149 | 0.099214 | 0.035028 | 0.256491 |
| of a different | 0.030828 | 0.442817 | 0.050282 | 0.135475 | | 0.060478 | 0.036297 | 0.055646 | | 0.045294 |
| precursor to the | | 0.058038 | 0.070672 | 0.063971 | | 0.062872 | | 0.046495 | | 0.056140 |
| transition state, | | 0.054012 | 0.061423 | 0.045588 | | 0.049047 | | 0.046513 | | 0.045318 |
| based in each | | 0.033991 | | 0.090480 | | 0.054034 | | 0.050048 | | 0.055720 |
| case on the | | | | 0.067227 | | 0.046588 | | 0.048978 | | 0.168430 |
| optimized starting | | | | 0.044884 | | 0.055280 | | 0.048104 | | 0.059298 |
| geometry of | | | | 0.057821 | | | | 0.056875 | | 8,418,276 |
| the first line | | | | | | | | 0.089174 | | |
| | | | | | | | | 0.057857 | | |
| | | | | | | | | 2,128,698 | | |
| | | | | | | | | 0.085683 | | |
| | | | | | | | | 0.037714 | | |
| Is a transition | Yes | No | No | No | No | Yes | No | Yes | No | No |
| state obtained? | | | | | | | | | | |

The values shown also include statistical extreme values, for example in columns 3.8 and 3.10.

TABLE A.4
Decarboxylation of acetoacetic acid

| | Each column shows a different optimized starting geometry | | | | | | | | | |
|---|---|---|---|---|---|---|---|---|---|---|
| Example | 4.1 | 4.2 | 4.3 | 4.4 | 4.5 | 4.6 | 4.7 | 4.8 | 4.9 | 4.10 |
| Gradient norm | 0.055244 | 0.055145 | 0.055285 | 0.055128 | 0.055106 | 0.055109 | 0.055218 | 0.055265 | 0.055413 | 0.055211 |
| B3 [Eₕ a₀⁻¹] of | | | | | | | | | | |
| the respective | | | | | | | | | | |
| optimized | | | | | | | | | | |
| starting geometry | | | | | | | | | | |
| Gradient norm | 0.049610 | 0.050850 | 0.057335 | 0.077653 | 0.060983 | 0.047224 | 0.050552 | 0.057350 | 0.058160 | 0.069370 |
| C3 [Eₕ a₀⁻¹], | 0.049458 | 0.062125 | 0.080891 | 0.058727 | 0.065141 | 0.067408 | 0.057388 | 0.621287 | 0.022109 | 0.082815 |
| where each line | 0.019246 | 0.039781 | 0.045796 | 0.043377 | 0.059457 | 0.112063 | 0.034994 | 0.052275 | | 0.087075 |
| represents the | | | 0.053588 | 0.071861 | 0.053003 | 0.051080 | | 0.056791 | | 0.113501 |
| gradient norm | | | 0.066665 | 0.069440 | 0.051556 | 0.055590 | | 0.072648 | | 0.051053 |
| of a different | | | 0.020932 | 0.077257 | 0.062801 | 0.073973 | | 0.045846 | | 0.048086 |
| precursor to the | | | | 0.045192 | 0.047389 | 0.087384 | | 0.047361 | | 0.051334 |
| transition state, | | | | 0.053762 | 0.113882 | 0.066149 | | 0.015354 | | 0.010003 |
| based in each | | | | 0.057251 | 0.069916 | 0.056958 | | | | |
| case on the | | | | 0.089196 | 0.061307 | 0.083602 | | | | |
| optimized starting | | | | 0.022387 | 0.045287 | 0.044368 | | | | |
| geometry of | | | | | 0.099284 | 0.042818 | | | | |
| the first line | | | | | 0.326467 | 0.054379 | | | | |
| | | | | | 0.053152 | 0.030123 | | | | |
| Is a transition | Yes | Yes | Yes | Yes | Yes | Yes | Yes | Yes | Yes | Yes |
| state obtained? | | | | | | | | | | |

TABLE A.5
Reduction of acetone to isobutene

| | Each column shows a different optimized starting geometry | | | | | | | | | |
|---|---|---|---|---|---|---|---|---|---|---|
| Example | 5.1 | 5.2 | 5.3 | 5.4 | 5.5 | 5.6 | 5.7 | 5.8 | 5.9 | 5.10 |
| Gradient norm | 0.070525 | 0.070525 | 0.070525 | 0.070525 | 0.070525 | 0.070525 | 0.070525 | 0.070525 | 0.070525 | 0.070525 |
| B3 [Eₕ a₀⁻¹] of | | | | | | | | | | |
| the respective | | | | | | | | | | |
| optimized | | | | | | | | | | |
| starting geometry | | | | | | | | | | |
| Gradient norm | 0.053417 | 0.039139 | 0.080632 | 0.051345 | 0.066935 | 0.067211 | 0.07307 | 0.070434 | 0.070324 | 0.086278 |
| C3 [Eₕ a₀⁻¹], | 0.032317 | | 0.086216 | 0.090356 | 0.047545 | 0.068587 | 0.071768 | 0.064323 | 0.071679 | 0.057452 |
| where each line | | | 0.055797 | 0.054938 | 0.026039 | 0.063053 | 0.054758 | 0.066577 | 0.052235 | 0.019717 |
| represents the | | | 0.065289 | 0.0546 | | 0.056315 | 0.059952 | 0.062511 | 0.061696 | |
| gradient norm | | | 0.052484 | 0.058829 | | 0.048515 | 0.061317 | 0.060599 | 0.036104 | |
| of a different | | | 0.043561 | 0.054373 | | 0.045782 | 0.069163 | 0.171984 | | |
| precursor to the | | | 0.057211 | 0.0659 | | 0.031459 | 0.05429 | 0.060208 | | |
| transition state, | | | 0.087309 | 0.044661 | | | 0.044025 | 0.066551 | | |
| based in each | | | 0.044804 | 0.283666 | | | 0.044025 | 0.067845 | | |
| case on the | | | 0.053187 | 0.054703 | | | 0.045235 | 0.057384 | | |
| optimized starting | | | 0.045268 | 0.043981 | | | 0.044025 | 0.06995 | | |
| geometry of | | | 0.057168 | 0.039904 | | | | 0.065572 | | |
| the first line | | | 0.053825 | | | | | 0.060108 | | |
| | | | 0.056278 | | | | | 0.060921 | | |
| | | | 0.084044 | | | | | 0.060597 | | |
| | | | 0.043251 | | | | | 0.045019 | | |
| | | | 0.043252 | | | | | 0.057638 | | |
| | | | 0.045823 | | | | | 0.065807 | | |
| | | | 0.039945 | | | | | 0.033495 | | |
| Is a transition | Yes | Yes | Yes | Yes | Yes | Yes | Yes | Yes | Yes | Yes |
| state obtained? | | | | | | | | | | |

### B EXAMPLES FOR ASCERTAINING A CATALYST FOR A CHEMICAL REACTION

In the examples that follow, the method according to the invention was used to examine which of the known catalysts 1,4-diazabicyclo[2.2.2]octane (DABCO) or N,N-dimethylaniline (DMA) is of better suitability for the hydrolysis of different isocyanates. This reaction, in the production of polyurethane foams, can be used to blow up the reaction mixture by the introduction of the gas formed, such that a foam is obtained.

In the examples, first of all, with the aid of the method according to the invention, the activation energies for the reaction of isocyanate and water in question were calculated and the values obtained were then compared with the results of laboratory experiments for the reactions calculated beforehand. The yardstick used for the activity of the catalysts tested in the laboratory experiments was the reaction temperature above which the catalysts showed activity. Whether the catalysts showed activity was determined in the laboratory experiment by the release of gases and the foaming of the reaction mixture.

The quantum-mechanical calculations were conducted with the Turbomole software package. The density functional theory method used was the TPSS density functional with a def2-SVP basis set, as implemented as standard in the Turbomole software package. For this purpose, the computer used was an Intel Xeon E5-2667v4 with 16-core processors with a clock frequency of 3.20 GHz and a 25 MB cache with 128 GB of DDR4 2400 rg ECC RAM.

For every reaction studied, a starting geometry was chosen as a starting point for the ascertaining of a transition state. The time taken for the calculation of the respective transition states was up to 8 hours.

The laboratory experiments were performed as follows:

A test tube with a magnetic stirrer was charged with 0.1 equivalent of catalyst, 1.0 equivalent of polyethylene glycol and 1.0 equivalent of water. The contents of the test tube were mixed thoroughly and 1.0 equivalent of the isocyanate in question was added while stirring. The temperature of the mixture was increased gradually with the aid of an oil bath with a thermostat until distinct evolution of gas was observable.

TABLE B.1
Molar masses and molar amount of the substances used.

| Component | M [g/mol] | n [mol] | m [mg] | Eq | d [g/ml | V [ml] |
|---|---|---|---|---|---|---|
| DABCO | 112.17 | 1.722 | 193 | 0.1 | — | — |
| DMA | 121.19 | 1.722 | 208 | 0.1 | 0.96 | 0.22 |
| TDI | 147.20 | 1.722 | 3000 | 1.0 | 1.22 | 2.46 |
| IPDI | 222.29 | 1.722 | 3828 | 1.0 | 1.06 | 3.61 |
| H₂O | 18.02 | 1.722 | 310 | 1.0 | 1.00 | 0.31 |
| PEG | 400 | 0.861 | 3444 | 0.5 | 1.128 | 3.05 |

### Example B.1: 1,4-Diazabicyclo[2.2.2]octane vs. N,N-dimethylaniline as Catalysts for the Hydrolysis of Toluene 1,4-diisocyanate

To evaluate the catalytic activity of 1,4-diazabicyclo[2.2.2]octane for catalysis of the hydrolysis of toluene 1,4-diisocyanate with water, the addition of water onto the isocyanate molecule was calculated by the method according to the invention for calculating transition states. For the geometry of the transition state ascertained by the method according to the invention, an activation energy of 12.8 kcal/mol was calculated. The same procedure was repeated for N,N-dimethylaniline (see table B.2), and an activation energy of 17.1 kcal/mol was calculated.

Under the experimental conditions described above, the reaction of toluene 1,4-diisocyanate with water in the presence of 1,4-diazabicyclo[2.2.2]octane as catalyst showed a significant reaction at 20° C. N,N-Dimethylaniline was tested as catalyst under identical conditions. A reaction temperature of 39° C. was needed here to be able to bring about an observable chemical reaction.

TABLE B.2
Compilation of experimental and simulated data

| | | | Temperature at | | |
|---|---|---|---|---|---|
| | | | which activation of | Gradient norm | |
| | | | the catalyst was | of the | |
| | | | observed in the | optimized | Gradient norm |
| | | Activation energy | laboratory | starting | of the |
| | | Simulated | experiment | geometry | transition state |
| Isocyanate | Catalyst | [kcal/mol] | [° C.] | [Eₕ a₀⁻¹] | [Eₕ a₀⁻¹] |
| TDI | DABCO | 12.8 | 20 | 0.067 | 0.042 |
| TDI | DMA | 17.1 | 39 | 0.068 | 0.030 |

This comparison shows that, with the aid of the method according to the invention, it is possible to calculate the molecular geometry of a transition state of a chemical reaction with sufficient precision that the activation energy which is calculated for that molecular geometry permits a meaningful conclusion as to the kinetics of the chemical reaction. The activation energy for DABCO calculated on the basis of the method according to the invention was much lower than that for DMA. The conclusion can be drawn from the results calculated that DABCO as catalyst for the hydrolysis of toluene 1,4-diisocyanate enables a transition state having a lower energy than when DMA is used as catalyst and that, consequently, in the performance of the reaction with DABCO as catalyst, less or no energy (proceeding from room temperature) has to be supplied for the reaction to proceed and products to be obtained.

These conclusions from the calculations are reflected in the results of the laboratory experiments. While the hydrolysis of toluene 1,4-diisocyanate with DABCO as catalyst already proceeded at a reaction temperature of 20° C., it was necessary to supply energy in the case of use of DMA, and the reaction did not run until 39° C.

### Example B.2: 1,4-Diazabicyclo[2.2.2]octane vs. N,N-dimethylaniline as Catalysts for the Hydrolysis of Isophorone Diisocyanate

To evaluate the catalytic activity of 1,4-diazabicyclo[2.2.2]octane for catalysis of the hydrolysis of isophorone diisocyanate with water, the addition of water onto the isocyanate molecule was calculated by the method according to the invention for calculating transition states. For the geometry of the transition state ascertained by the method according to the invention, an activation energy of 16.3 kcal/mol was calculated.

The same procedure was repeated for N,N-dimethylaniline (see table B.3), and an activation energy of 19.9 kcal/mol was calculated.

Under the experimental conditions described, the reaction of isophorone diisocyanate with water in the presence of 1,4-diazabicyclo[2.2.2]octane as catalyst showed a significant reaction at 31° C.

N,N-Dimethylaniline was tested as catalyst under identical conditions. A reaction temperature of 116° C. was needed here to be able to bring about an observable chemical reaction.

TABLE B.3
Compilation of experimental and simulated data

| | | | Temperature at | Gradient norm | |
|---|---|---|---|---|---|
| | | | which activation of | of the | Gradient norm |
| | | | the catalyst was | optimized | of the |
| | | Activation energy | observed in the | starting | transition |
| | | Simulated | laboratory | geometry | state |
| Isocyanate | Catalyst | [kcal/mol] | experiment [° C.] | [Eₕ a₀⁻¹] | [Eₕ a₀⁻¹] |
| IPDI | DABCO | 16.3 | 31 | 0.067 | 0.043 |
| IPDI | DMA | 19.9 | 116 | 0.067 | 0.045 |

This comparison shows that, with the aid of the method according to the invention, it is possible to calculate the molecular geometry of a transition state of a chemical reaction with very little user effort and sufficient precision that the activation energy which is calculated for that molecular geometry permits a meaningful conclusion as to the kinetics of the chemical reaction. The activation energy for DABCO calculated on the basis of the method according to the invention was much lower than that for DMA. The conclusion can be drawn from the results calculated that DABCO as catalyst for the hydrolysis of toluene 1,4-diisocyanate enables a transition state having a lower energy than when DMA is used as catalyst and that, consequently, in the performance of the reaction with DABCO as catalyst, less or no energy (proceeding from room temperature) has to be supplied for the reaction to proceed and products to be obtained.

These conclusions from the calculations are reflected in the results of the laboratory experiments. While the hydrolysis of toluene 1,4-diisocyanate with DABCO as catalyst already proceeded at a reaction temperature of 31° C., it was necessary to supply much more energy in the case of use of DMA, and the reaction did not run until 116° C.

1. A computer-implemented method of calculating transition states of a chemical reaction, comprising the steps of;
A Generating a starting geometry by
A1 providing the three-dimensional representation of at least one molecule at ground state energy,
A2 selecting at least one bond of the at least one molecule and selecting a length of the bond, where the selected length does not correspond to the length of the bond at ground state energy of the molecule, such that a starting geometry for the chemical reaction is obtained, and
A3 representing the starting geometry in three dimensions in Cartesian and/or internal coordinates,
B Ascertaining an optimized starting geometry by
B1 defining a function space encompassing the at least one bond from step A2 and the atoms joined by this bond,
B2 optimizing the geometry of the starting geometry on the basis of the function space selected in step B1 by means of a quantum-chemical method and with the boundary condition that the length of the at least one bond selected in step A2 is kept constant, such that an optimized starting geometry is obtained,
B3 ascertaining the gradient norm B3 for the optimized starting geometry, where the gradient norm is obtained via the first derivative of a function E=f(x) by means of the quantum-chemical method, with E=total energy of the optimized starting geometry and x=nuclear coordinates of the molecule in the optimized starting geometry, and
B4.1 when the gradient norm B3 ∇ is 0≤∇≤0.07 Eₕ a₀⁻¹, classifying the optimized starting geometry as a precursor to the transition state of the chemical reaction and continuing the method with step D1, or
B4.2 when the gradient norm B3 ∇ is >0.07 Eₕ a₀⁻¹, ascertaining the precursor to the transition state of the chemical reaction proceeding from the optimized starting geometry by a method comprising the following steps:
C Ascertaining the precursor to the transition state of the chemical reaction by
C1 varying the optimized starting geometry using a Monte Carlo algorithm,
C1.1 wherein at least one atom from the function space selected in step B1 is selected at random,
C1.2 wherein a vector for a deflection of the atom chosen in step C1.1 is selected at random,
C1.3 wherein the atom selected in step C1.1 is deflected from its position in the optimized starting geometry using the vector from step C1.2, so as to obtain a precursor to the transition state of the chemical reaction, and
C2 optimizing the geometry of the precursor to the transition state by means of the quantum-chemical method and with the boundary condition that the at least one bond from step A2 has the length that was ascertained in step C1.3, and
C3 ascertaining the gradient norm C3 for the precursor to the transition state from step C2, where the gradient norm is obtained via the first derivative of the function E=f(x) by means of the quantum-chemical method, with E=total energy of the precursor to the transition state and x=nuclear coordinates of the molecule in the precursor to the transition state, and
C4.1 when the gradient norm C3 ∇ is 0≤∇≤0.07 Eₕ a₀⁻¹, continuing the method with step D1, or
C4.2 when the gradient norm C3 ∇ is >0.07 Eₕ a₀⁻¹, repeating steps C1 to C3 until a gradient norm C3 of 0≤∇≤0.07 Eₕ a₀⁻¹ is obtained, wherein
(a) if steps C1 to C3 have been performed once, the geometry of the precursor to the transition state is varied in step C1 when the value of its gradient norm C3 is lower than the value of the gradient norm B3 of the optimized starting geometry, or
(b) if steps C1 to C3 have been performed more than once, the geometry of the optimized starting geometry or that precursor to the transition state from the preceding repetitions that has the lowest value for the gradient norm C3 or B3 compared to all the gradient norms C3 and B3 previously obtained is varied in step C1,
D Ascertaining the transition state by
D1 relaxing the precursor from step C4.1 or the precursor from step B4.1 by means of the quantum-chemical method and a pseudo-Newton-Raphson algorithm, such that the transition state is obtained, and
D2 optionally ascertaining an equilibrium state by deflecting the transition state, such that a deflected transition state is obtained, and relaxing the deflected transition state by means of the quantum-chemical method, such that an equilibrium state is obtained.

2. The method of claim 1, wherein the quantum-chemical method from steps B2, B3, C2, C3, D1 and D2 is a semiempirical method, density functional theory method or an approximation of the Schrödinger equation.

3. The method claim 1, wherein the chemical reaction is a synthesis selected from the group consisting of polymer syntheses, polyurethane syntheses, syntheses of monomers for polymerization reactions, industrially required commodity chemicals, additives, surfactants and active pharmacological ingredients.

4. The method of claim 1, wherein, in step A1, at least two molecules I and II are provided and, in step A2, alternatively or additionally to the at least one bond, at least one distance between at least one atom from molecule I and at least one atom from molecule II and the length of the at least one distance may also be selected, where the length of the distance is especially not more than 230 pm.

5. The method of claim 4, wherein molecule I is a catalyst for the chemical reaction, and molecule II is a reactant in the chemical reaction.

6. The method of claim 4, wherein molecule I has a size of ≤100 atoms and molecule II has a size of ≤100 atoms, and the sum total of the atoms from molecule I and from molecule II should preferably be ≤100 atoms.

7. The method of claim 1, wherein information about the transition state ascertained according to step D.1 or the equilibrium state ascertained according to step D.2 is communicated to a user.

8. The method of claim 1, wherein information about the transition state ascertained according to step D.1 and/or the equilibrium state ascertained according to step D.2 is received by a user.

9. The method of claim 5, wherein the molecule I is synthesized after step D.1 or after step D.2.

10. The method of claim 5, wherein under a chemical reaction with the molecule I as catalyst is performed after step D.1 or after step D.2.

11. The method of claim 5, wherein under a chemical reaction with the molecule II as co-reactant is performed after step D.1 or after step D.2.

12. A system for data processing, comprising means of executing a method comprising the steps of:
A Generating a starting geometry by
A1 providing the three-dimensional representation of at least one molecule at ground state energy,
A2 selecting at least one bond of the at least one molecule and selecting a length of the bond, where the selected length does not correspond to the length of the bond at ground state energy of the molecule, such that a starting geometry for the chemical reaction is obtained, and
A3 representing the starting geometry in three dimensions in Cartesian and/or internal coordinates,
B Ascertaining an optimized starting geometry by
B1 defining a function space encompassing the at least one bond from step A2 and the atoms joined by this bond,
B2 optimizing the geometry of the starting geometry on the basis of the function space selected in step B1 by means of a quantum-chemical method and with the boundary condition that the length of the at least one bond selected in step A2 is kept constant, such that an optimized starting geometry is obtained,
B3 ascertaining the gradient norm B3 for the optimized starting geometry, where the gradient norm is obtained via the first derivative of a function E=f(x) by means of the quantum-chemical method, with E=total energy of the optimized starting geometry and x=nuclear coordinates of the molecule in the optimized starting geometry, and
B4.1 when the gradient norm B3 ∇ is 0≤∇≤0.07 Eₕ a₀⁻¹, classifying the optimized starting geometry as a precursor to the transition state of the chemical reaction and continuing the method with step D1, or
B4.2 when the gradient norm B3 ∇ is >0.07 Eₕ a₀⁻¹, ascertaining the precursor to the transition state of the chemical reaction proceeding from the optimized starting geometry by a method comprising the following steps:
C Ascertaining the precursor to the transition state of the chemical reaction
C1 varying the optimized starting geometry using a Monte Carlo algorithm,
C1.1 wherein at least one atom from the function space selected in step B1 is selected at random,
C1.2 wherein a vector for a deflection of the atom chosen in step C1.1 is selected at random,
C1.3 wherein the atom selected in step C1.1 is deflected from its position in the optimized starting geometry using the vector from step C1.2, so as to obtain a precursor to the transition state of the chemical reaction, and
C2 optimizing the geometry of the precursor to the transition state by means of the quantum-chemical method and with the boundary condition that the at least one bond from step A2 has the length that was ascertained in step C1.3, and
C3 ascertaining the gradient norm C3 for the precursor to the transition state from step C2, where the gradient norm is obtained via the first derivative of the function E=f(x) by means of the quantum-chemical method, with E=total energy of the precursor to the transition state and x=nuclear coordinates of the molecule in the precursor to the transition state, and
C4.1 when the gradient norm C3 ∇ is 0≤∇≤0.07 Eₕ a₀⁻¹, continuing the method with step D1, or
C4.2 when the gradient norm C3 ∇ is >0.07 Eₕ a₀⁻¹, repeating steps C1 to C3 until a gradient norm C3 of 0≤∇≤0.07 Eₕ a₀⁻¹ is obtained, wherein
(a) if steps C1 to C3 have been performed once, the geometry of the precursor to the transition state is varied in step C1 when the value of its gradient norm C3 is lower than the value of the gradient norm B3 of the optimized starting geometry, or
(b) if steps C1 to C3 have been performed more than once, the geometry of the optimized starting geometry or that precursor to the transition state from the preceding repetitions that has the lowest value for the gradient norm C3 or B3 compared to all the gradient norms C3 and B3 previously obtained is varied in step C1,
D Ascertaining the transition state by
D1 relaxing the precursor from step C4.1 or the precursor from step B4.1 by means of the quantum-chemical method and a pseudo-Newton-Raphson algorithm, such that the transition state is obtained, and
D2 optionally ascertaining an equilibrium state by deflecting the transition state, such that a deflected transition state is obtained, and relaxing the deflected transition state by means of the quantum-chemical method, such that an equilibrium state is obtained.

13.-15. (canceled)

16. The system of claim 12, wherein the quantum-chemical method from steps B2, B3, C2, C3, D1 and D2 is a semiempirical method, density functional theory method or an approximation of the Schrödinger equation.

17. The system of claim 12, wherein the chemical reaction is a synthesis selected from the group consisting of polymer syntheses, polyurethane syntheses, syntheses of monomers for polymerization reactions, industrially required commodity chemicals, additives, surfactants and active pharmacological ingredients.

18. The system of claim 12, wherein, in step A1, at least two molecules I and II are provided and, in step A2, alternatively or additionally to the at least one bond, at least one distance between at least one atom from molecule I and at least one atom from molecule II and the length of the at least one distance may also be selected, where the length of the distance is especially not more than 230 pm.

19. The system of claim 18, wherein molecule I is a catalyst for the chemical reaction, and molecule II is a reactant in the chemical reaction.

20. The system of claim 18, wherein molecule I has a size of ≤100 atoms and molecule II has a size of ≤100 atoms, and the sum total of the atoms from molecule I and from molecule II should preferably be ≤100 atoms.

21. The system of claim 12, wherein information about the transition state ascertained according to step D.1 or the equilibrium state ascertained according to step D.2 is communicated to a user.

22. The method of claim 12, wherein information about the transition state ascertained according to step D.1 and/or the equilibrium state ascertained according to step D.2 is received by a user.

23. The system of claim 19, wherein the molecule I is synthesized after step D.1 or after step D.2.
